(19) United States
(12) Patent Application Publication
(10) Pub. No.: US 2009/0215671 A1
(43) Pub. Date: Aug. 27, 2009

(54) Compositions And Methods For Treatment of Neural Disorders Using Transforming Growth Factor-Beta Superfamily Proteins And Their Antagonists

(75) Inventors: Anne L. Calof, Laguna Beach, CA (US); Joon Kim, Irvine, CA (US); Shimako Kawauchi, Costa Mesa, CA (US)

Correspondence Address: FISH & ASSOCIATES, PC;ROBERT D. FISH, 2603 Main Street, Suite 1000, Irvine, CA, 92614-6232, US

(73) Assignee: THE REGENTS OF THE UNIVERSITY OF CALIFORNIA, Oakland, CA (US)

(21) Appl. No.: 11/915,665

(22) PCT Filed: May 30, 2006
(86) PCT No.: PCT/US2006/021016

§ 371 Date: Apr. 8, 2009

Related U.S. Application Data
(60) Provisional application No. 60/685,630, filed on May 27, 2005.

(51) Int. Cl. A61K 38/22; C12N 5/00; A61P 27/16; A61P 27/06; A61P 27/02

(52) U.S. Cl. 514/8; 435/377; 514/12

(57) ABSTRACT

Contemplated compositions and methods employ a TGF-beta superfamily protein or antagonist thereof to treat a neural disorder characterized by an imbalance in differentiated functional sensory and neural cells derived from a sensory/neural progenitor cell. Preferably, GDF-11 and/or antagonists thereof are employed in the treatment of diseases in which visual and/or auditory progenitor cells will provide for a repair mechanism to the disease. Most preferably, GDF-11 is employed as a modulator of competency to increase production of retinal ganglion cells, retinal photoreceptors, retinal amacrine cells, sensory hair and supporting cells of the vestibulocochlear epithelium, and/or neurons and supporting cells of the spiral acoustic ganglion and vestibulo-cochlear (auditory) nerve to which it gives rise.

This application claims priority to our copending U.S. provisional patent application with the Ser. No. 60/685,630, which was filed May 27, 2005.

This invention was made with government support from the NIH, grant numbers NIH 2 R01 DC03583-06 and NIH P01 HD38761. The government may have certain rights in the invention.

### FIELD OF THE INVENTION

The field of the invention is transforming growth factor-beta (TGF-beta) superfamily proteins and their antagonists, especially as they relate to treatment of neural disorders.

### BACKGROUND OF THE INVENTION

Many neural disorders eventually result in in-evocable loss of function of the affected organ, and in most cases, only few treatment options are currently available for such diseases. For example, while progression of macular degeneration can at least in some cases be slowed down, complete restoration of sight is often not realized as the defective tissue structure is not replaced by healthy tissue. Similarly, sensorineural hearing loss is generally not reversed as the hair cells are not completely regenerated. Recent developments suggest that vision and hearing may be restored to at least some degree using implanted devices. Unfortunately, the connectivity of such devices with the appropriate neural tissue is often problematic, and the visual/auditory perception using such implants is generally insufficient to allow reading, hand-eye fine-coordination, or to participate in an ordinary conversation. Other reports have suggested that loss of visual and/or auditory function may be restored using stem cell based therapy. However, most of currently known stem cell technologies either reply on embryonic stem cells that are problematic from numerous perspectives, and/or require animal serum and feeder layers that prevent human use. Even where such difficulties are not encountered, the proper induction conditions for stem cells to produce the desired cell type for repair are often elusive.

Therefore, while there are some devices, compositions, and methods for improving or restoration of neural function are known in the art, all or almost all of them suffer from one or more disadvantages. Consequently, there is still a need to provide improved compositions and methods to restore neural, and especially ocular and auditory function.

### SUMMARY OF THE INVENTION

The present invention is directed to compositions and methods in which transforming growth factor-beta (TGF-beta) superfamily proteins and their antagonists are employed as therapeutic modalities in vivo and/or in vitro to influence progenitor cells to thereby restore lost neural cell function and/or to counterbalance an imbalance among cell types that derived from such progenitor cells. More specifically, the inventors discovered that various TGF-β superfamily proteins and their antagonists can be employed as modifiers of susceptibility to developmental stimuli in progenitor cells to thereby treat diseases in which progenitor cells and their differentiated daughter cells contribute to the disease.

In one aspect of the inventive subject matter, a method of enabling modulation of susceptibility of a neural progenitor cell to a developmental stimulus includes one step in which a composition is provided that includes at least one of a GDF-11, a GDF-11 analog, and a GDF-11 antagonist in a pharmaceutically acceptable formulation. In another step, information is provided to administer the composition to the neural progenitor cell at a dosage and under a protocol effective to modulate the susceptibility (differentiation and/or function) of the neural progenitor cell. Most typically, the modulation of the susceptibility is maintained under the protocol in such methods for a period effective to increase or decrease a number of differentiated daughter cells derived from the neural progenitor cell.

While not limiting to the inventive subject matter, it is generally preferred that the neural progenitor cell is a progenitor cell for cells associated with visual or auditory function. Therefore, contemplated neural progenitor cells will preferably include cells giving rise to cells of the neural retina, including retinal ganglion cells, amacrine cells, rod and cone photoreceptor cells, horizontal cells, bipolar cells, and Muller glia cells. Contemplated progenitor cells will further include those giving rise to cells of the primary auditory pathway, including inner and outer hair cells of the vestibulo-cochlear epithelium, neurons and glia of the spiral-acoustic ganglion, and the vestibulo-cochlear nerve. Depending on the particular type of progenitor cell, modulation of the susceptibility may be mediated by expression of genes encoding one or more transcription factors whose function confers neural and/or sensory functional competence and/or identity. For example, visual progenitor cells may express the Math5 gene, while auditory progenitor cells may express the Math1 gene and/or Neurogenin-1.

It is furthermore contemplated that administration of GDF-11 and/or a GDF-11 analog will result in an decrease of retinal ganglion cells (RGCs) derived from a visual neural progenitor cell (with a possible increase in photoreceptors and/or amacrine cells), whereas administration of a GDF-11 antagonist (e.g., follistatin) will result in an increase of retinal ganglion cells (with a possible decrease in photoreceptors and/or amacrine cells) derived from these progenitor cells. It should be further noted that the administration may be in vivo (e.g., via injection, viral vector, transfection, etc.) or in vitro. In such case, the administration may be directly to the progenitor cell and/or to a stem cell that is developmentally upstream of the progenitor cell (e.g., totipotent stem cell, pluripotent stem cell, germ line lineage stem cell, endodermal, mesodermal, ectodermal stem cell).

Especially contemplated GDF-11 antagonist include follistatin, and contemplated GDF-11 analogs include GDF-8, activin beta A and activin beta B. Furthermore, the GDF-11, the GDF-11 analog, and/or the GDF-11 antagonist may be native and isolated from a biological source, or recombinant or produced in situ in neural tissue (e.g., via transfection). Therefore, at least one of the GDF-11, the GDF-11 analog, and the GDF-11 antagonist may be produced from a viral genome.

In another aspect of the inventive subject matter, a pharmaceutical kit for treatment of a neural disorder that results from changes in follistatin function and/or function of other antagonists of TGF-beta superfamily proteins, will include at least one of a GDF-11, a GDF-11 analog, and a GDF-11 antagonist in a pharmaceutically acceptable formulation. Contemplated kits will further include an instruction that is associated with the formulation (e.g., as packing insert, package label, etc.) wherein the instruction pertains to administration of the formulation to a sensory or neural progenitor cell at a dosage and under a protocol effective to modulate the susceptibility of the neural progenitor cell. In such instructions, the protocol is descriptive of a protocol effective to maintain modulation of the susceptibility for a period sufficient to increase/decrease a number of functional sensory and/or neural cells derived from the neural progenitor cell. The term “neural” as used herein refers to neural cells as well as to cells that are involved in the sensory apparatus (e.g., in the eye, ciliary body cells, retinal pigmented epithelium (RPE) cells, Muller glia, etc.; and in the primary auditory pathway, inner and outer hair cells of the cochlear and/or vestibular epithelia, supporting cells of the vestibulo-cochlear epithelium, and neurons and supporting cells of the spiral-acoustic ganglion and/or vestibulo-cochlear nerve, etc.). Therefore, the term “neural” cell also refers to “sensory” cells and is also used interchangeably with the terms “neural/sensory” or “neural and/or sensory” herein.

Most preferably, the GDF-11 analog is GDF-8, activin beta A or activin beta B, and the GDF-11 antagonist is follistatin. Similar to the method above, it is contemplated that in at least some cases the modulation of susceptibility is described as a modulation of expression of the Math5, Math1, or Neurogenin 1 gene. It is further preferred that the progenitor cell is a progenitor cell for cells associated with visual or auditory function. Thus, especially contemplated differentiated neural cells resulting from treatment include retinal ganglion cells, amiacrine cells, photoreceptor cells, inner and outer hair cells, and spiral-acoustic ganglion neurons.

Therefore, in a still further aspect of the inventive subject matter, the inventors also contemplate use of at least one of a GDF-11, a GDF-11 analog, and a GDF-11 antagonist in the manufacture of a medicament for treatment of an auditory or visual neural disorder, wherein the disorder is a follistatin-responsive disease (wherein at least one of the GDF-11, the GDF-11 analog, and the GDF-11 antagonist can be a recombinant protein). Preferably, the follistatin-responsive disease is characterized by exacerbation of the disease state upon administration of compounds that elevate or reduce the amount of follistatin present in the body. The terms “follistatin responsive disease”, “disorder is follistatin responsive”, and “disorder that is characterized in responsiveness to follistatin” refer to a sensory and/or neurological disease or disorder associated with a change in follistatin function and or quantity, and also refer to a disease or disorder associated with a change in function/quantity of other protein antagonists of TGF-beta superfamily ligands in sensory and/or neural structures. Therefore, contemplated disorders include macular degeneration, photoreceptor degeneration, retinal ganglion cell degeneration, Leber's congenital amaurosis, and sensorineural hearing loss.

Various objects, features, aspects and advantages of the present invention will become more apparent from the following detailed description of preferred embodiments of the invention.

### BRIEF DESCRIPTION OF THE DRAWINGS

FIGS. 1A-1E are photomicrographs and bar graphs illustrating GDF-11 and Fst expression in the eye at various times and in selected wildtype and mutant mice, and effects absence of GDF-11 on retinal ganglion cell number and optic nerve size.

FIG. 2 is a photomicrograph depicting expression of various types of GDF-11 receptor during retinal development.

FIG. 3A is a photomicrograph depicting developmental differences in wildtype and selected mutant mice and FIG. 3B is a graph representing quantification of neurofilament-stained optic nerve sections shown in FIG. 1E.

FIGS. 4A-4H are photomicrographs of normal cell proliferation patterns in Gdf11^{tm2/tm2} and Fst^{-/-} retinas and corresponding quantitative analysis of the phosphorylated histone H3.

FIGS. 5A-5E are microphotographic and quantitative representations of the developmental differences in differentiation of retinal ganglion cells in Gdfp11 null, Fst null and wildtype mice.

FIGS. 6A-6C are microphotographs illustrating expression of key regulatory and ventral patterning genes in retinas of wildtype and mutant mice.

FIG. 7 is a graph depicting differences in Brn3b⁺ cells (retinal ganglion cells) among wildtype and various mutant strains.

FIGS. 8A and 8B are photomicrographs illustrating differential expression of selected markers in wildtype and mutant/treated retinas.

FIGS. 9A-9E are photomicrographs depicting express of key regulatory genes in wildtype and mutant/treated retinas.

FIGS. 10A and 10B are photomicrographic and quantitative representations of Lin1 expression, a marker for retinal horizontal cells, in wildtype and mutant retinas.

FIGS. 11A-11C are photomicrographs depicting expression patterns of transcription factors implicated in retinal neurogenesis in wildtype and mutant retinas.

FIG. 12A is an autoradiograph and 12B is the corresponding quantitative analysis of p27Kip1, a cell cycle regulator protein, in wildtype and mutant retinas.

### DETAILED DESCRIPTION

The inventors have discovered that various compounds and compositions that interact with signaling pathway(s) that are functionally associated with selected members of the TGF-beta superfamily of signaling proteins can be employed to regulate production of neural and sensory tissue, and especially auditory and visual neural tissue, wherein at least in some cases regulation is achieved by changing the susceptibility sensory/neural progenitor cells to developmental stimuli rather than by changing proliferation of progenitors cell.

Thus, and viewed from another perspective, it should be appreciated that modification of susceptibility to developmental stimuli can be employed as a modality to treat diseases in which progenitor cells and their sensory and neural daughter cells contribute to the disease. Using such approach, compositions and methods are contemplated that identify modifiers of susceptibility to sensory and neural differentiation. Once identified, such compounds and their analogs can then be employed to influence progenitor cells to give rise to increased or decreased quantities of one or more differentiated sensory and/or neural daughter cell types. Alternatively, or additionally, compounds may also be identified that interact with one or more pathways that are associated with the regulation of neural tissue. For example, where progenitor cells are neural progenitor cells for the development and/or repair of auditory and/or visual neural tissue, TGF-beta superfamily proteins (e.g., GDF-11 proteins and analogs or antagonists thereof) can be used as modifier of susceptibility to sensory and/or neural differentiation.

In one particularly preferred example, the inventors discovered that secreted growth and differentiation factor 11 (GDF-11) controls the number of retinal ganglion (RGC) cells as well as amacrine and photoreceptor cells without substantially affecting proliferation of their progenitor cells (i.e., changing proliferation less than 10% abs.), which is entirely contrary to the known cytostatic (inhibition of proliferation) effect on proliferation in other tissues. Remarkably, it was found that the number of RGCs is controlled by regulating the duration of expression of the regulatory gene Math5, which confers competence on progenitor cells to develop to an RGC. In a further and closely related example, and based on the observations with retinal progenitor cells and other data (infra), it is contemplated that GDF-11 also influences progenitor cells in vestibulo-cochlear sensory epithelium that contains sensory hair cell progenitors. In this example, it is contemplated that hair cell formation can be decreased by exposure of the auditory progenitor cells to GDF-11. The mechanism underlying the modification of susceptibility in auditory progenitor cells is contemplated to involve Math1, which encodes a protein that is thought to be a functional analog (basic helix-loop-helix transcription factor) of Math5 in visual progenitor cells. Similarly, and in yet another example, auditory progenitor cells can be modified in their susceptibility to differentiation to produce increased/decreased amounts of spiral acoustic ganglion cells and/or vestibulo-cochlear nerve cells via up-/downregulation of Neurogenin1, another basic helix-loop-helix transcription factor that is required for development of spiral-acoustic ganglion neurons. Thus, it is contemplated that GDF-11 may in general regulate expression of basic helix-loop-helix transcription factors in neural progenitor cells and with that affect sensory/neural differentiation, susceptibility to same, and in some cases also cell proliferation of such progenitor cells. On a molecular level, it should be appreciated that all genes affected by the expression of contemplated basic helix-loop-helix transcription factors will be regulated by TGF-beta superfamily proteins, and especially GDF-11 proteins and analogs or antagonists thereof. Consequently, and in yet another preferred aspect of the inventive subject matter, it should be appreciated that GDF-11 and analogs thereof may serve as a competency modulator for development of a progenitor cell to a more differentiated sensory/neural cell, and that GDF-11 and its analogs may therefore govern relative numbers of distinct and downstream differentiated sensory/neural cell populations obtained from a progenitor cell population.

With respect to suitable TGF-β superfamily proteins contemplated herein, it should be appreciated that while GDF-11 is a preferred compound, numerous alternative compounds are also deemed suitable so long as such compounds interact with one or more components in a signaling pathway functionally associated with selected members of the TGF-β superfamily. Thus, and viewed from one perspective, chemically (e.g., pegylated, acylated, etc.) and/or biologically (e.g., mutated, truncated, fused, enzymatically modified, etc.) modified versions of GDF-11 may be suitable, as well as GDF-11 analogs from a species other than human. Viewed from another perspective, suitable GDF-11 alternatives also include those molecules that yield at least a moderate signal response in a GDF-11 associated pathway (e.g., 10% of the influence on Math5 expression in a human progenitor cell relative to human GDF-11 influence in that cell). Consequently, GDF-11 homologs, analogs, or otherwise related forms are especially contemplated herein. For example, GDF-8 may replace GDF-11 in at least some instances. Viewed from yet another perspective, all molecules other than GDF-11 may also be suitable that bind to receptor/binding sites to which GDF-11 is known to bind (e.g., Activin type IIA and IIB receptors, etc). For example, suitable molecules may be identified by their interaction with the GDF-11 receptors, resulting in phosphorylation of Smad 2 or 3 in the cytoplasm of the responding cell. Among other compounds, TGF beta 1, TGF beta 2, TGF beta 3, GDF-8, Nodal, and all activins are therefore especially contemplated. Still further, it should be noted that (typically synthetic) small molecule GDF-11 agonists are also contemplated for use herein (see e.g., Nature Reviews—Drug Discovery (2004) Vol. 3, p 111-22).

Alternatively, and especially where the opposite effect of GDF-11 stimulation is desired (e.g., downregulation of susceptibility for differentiation), it should be appreciated that numerous TGF-β antagonists may be employed to either downregulate or even block GDF-11 mediated effects. There Core, and among other suitable compounds, particularly preferred compounds include follistatin, FLRG (FLRP3), GASP 1, GASP2, and related TGF-beta family members, and other natural or synthetic antagonists of GDF-11 and/or GDF-8. Typically, suitable GDF-11 antagonists may operate in one or more manners, including competitive or allosteric receptor blocking, cross-modulation from an upstream and/or downstream component in the same pathway, sequestration and/or binding of GDF-11, etc. Therefore, viewed from a different perspective, GDF-11 antagonists may be characterized as proteins (e.g., secreted or membrane-associated) that also antagonize the above mentioned of GDF-11 analogs. Such molecules may include recombinant proteins as well as synthetic small-molecule drugs (e.g., acting on cytoplasmic signaling pathways of GDF-11 and its analogs).

Still further, it should be recognized that contemplated GDF-11 binding effects or GDF-11 antagonist action may also be precipitated in a GDF-11/antagonist-independent manner in which an up- and/or downstream component in the GDF-11 associated pathway is targeted. For example, where GDF-11 effects should be suppressed or reduced, upstream components in that pathway may be muted or subdued. On the other hand, where it is desired that the GDF-11 mediated effect is to be amplified, downstream components may be targeted to amplify such signals (e.g., via recombinant introduction of constitutively active kinases, overexpression of associated kinases, etc.). Similarly, expression and/or secretion of GDF-11 and/or its binding sites (e.g., Activin type IIA and IIB receptors) may be enhanced or subdued using technologies well known in the art (e.g., antisense or siRNA, knockout/knockdown mutations, etc.).

In another particularly preferred aspect and based on the inventors' findings, the inventors contemplate that the compounds and compositions discussed above may be employed as a therapeutic and/or prophylactic modality to treat various disorders that may be characterized by anatomical and/or functional decline/loss of neural tissue. For example, contemplated neural visual disorders include those due to a loss of photoreceptors, disorders associated with dysfunction or loss of amacrine cells, and retinal ganglion degenerations. Thus, and among other things, contemplated disorders particularly include macular degeneration (age-related or otherwise), Leber's congenital amaurosis, and glaucoma- or ischemia-induced retinal ganglion degeneration, etc. In another example, contemplated auditory disorders will include those associated with dysfunction or loss of hair cells of the vestibulo-cochlear epithelium, spiral acoustic ganglion cells, and/or vestibulo-cochlear nerve cells.

In the treatment and/or prophylaxis of such diseases, it is generally preferred that the compounds or compositions according to the inventive subject matter are formulated in a pharmaceutically acceptable manner. Suitable formulations will preferably include liquid preparations for injection into the anterior and/or posterior chamber of the eye, or for injection into the semicircular canals, cochlea, and/or bony labyrinth of the temporal bone. Alternatively, or additionally, implantable carriers (e.g., biodegradable/dissolving) may be formulated such that the carrier comprises therapeutically effective amounts of the compound or composition, and that the carrier can release the compound or composition in a controlled and predetermined manner. Among other suitable carriers, the release may be time-dependent and/or initiated by irradiation with light of one or more wavelengths.

It is contemplated that pharmaceutical compositions according to the inventive subject matter comprise at least one of contemplated compounds (e.g., one or more GDF-11, GDF-11 analog, and/or GDF-11 antagonist) together with a pharmaceutically acceptable carrier. Depending on the particular use, it should be recognized that formulation, route, and/or administration schedule may vary considerably, and it is generally contemplated that the specific formulation, route, and/or administration is not limiting to the inventive subject matter.

Therefore, appropriate formulations include formulations for oral, parenteral, and/or topical (including nasal, buccal, and sublingual) administration, and it is further preferred that contemplated formulations are in unit dosage form. It is still further preferred that the amount of the contemplated compound (active ingredient) that is combined with a carrier to form a unit dosage form will be the amount that produces a therapeutic effect. Thus, with respect to the amount of the TGF-β type agonists and/or antagonists administered to the neural target tissue, it is contemplated that suitable amounts include those precipitating at least a 2-5%, and more typically at least 5-10% absolute deviation as compared to a control experiment without addition of the TGF-β type agonist and/or antagonist. Suitable amounts will therefore be in the range of about 0.1 ng to 1.0 mg per dosage unit, more typically between about 10 ng to 100 microgram per dosage unit, and most typically between about 100 ng to 10 microgram per dosage unit. Depending on the formulation, the percentage (% wt) of the active ingredient will typically range from about 0.001 percent to about ninety-nine percent of the total weight, more preferably from about 0.01 percent to about 70 percent, and most preferably from about 0.01 percent to about 50 percent.

It should be appreciated, however, that the administered dose of the pharmaceutical composition will vary considerably, and a particular dose will at least in part depend on (a) the amount of active ingredient which is effective to achieve a desired therapeutic response, (b) the formulation of contemplated compounds, (c) the route of administration, (d) the pharmacokinetic and pharmnacodynamic property of the particular compound, and (e) other factors, including age, sex, weight, general health, and prior medical history of the patient being treated. A person of ordinary skill in the art can readily determine and prescribe the effective amount of the pharmaceutical composition required. For example, a physician could start dosing a patient at levels lower than normally required for a desired therapeutic effect and then increase the dosage until the desired effect is achieved.

It is generally preferred that the daily dose of contemplated compounds will typically correspond to the amount of the compound which is the lowest dose effective to produce a desired therapeutic effect. Such an effective dose will generally depend upon the factors described above. Therefore, doses of the compounds according to the inventive subject matter will range from about 0.001 mg to about 1 00 mg per kilogram of body weight per day, more preferably from about 0.01 to about 50 mg per kg per day, and still more preferably from about 0.1 to about 40 mg per kg per day. Thus, a unit dose of contemplated compounds will range from about 0.01 mg to about 5000 mg, more preferably from about 0.01 mg to about 500 mg, and most preferably from about 0.1 mg to about 100 mg. If desired, the effective daily dose of the active compound may be administered as two, three, four, five, six or more sub-doses administered separately at appropriate intervals throughout the day, optionally, in unit dosage forms.

Viewed from another perspective, a unit close of the contemplated compounds will preferably be an amount sufficient to modulate the susceptibility of the neural progenitor cell. In especially preferred embodiments, a unit dose will be selected from an amount sufficient to increase Smad2/3 phosphoryltion levels and/or the expression of a basic helix-loop-helix transcription factor (most typically Math1, Math5, and/or Neurogenin 1) by at least 10% and more typically at least 20% (absolute an-d/or on a temporal basis) over pre-administration levels.

It is generally contemplated that the compounds according to the inventive subject matter may be prepared in a formulation for parenteral use, and especially contemplated parenteral formulations will be liquid formulations for injection. Therefore, appropriate formulations will generally include a pharmaceutically acceptable solvent (e.g., sterile isotonic aqueous or non-aqueous solution), and may be prepared as a dispersion, suspension, or emulsion. Alternatively, parenteral formulations may also be provided as a kit that includes contemplated compounds and other components that may be reconstituted to a liquid product prior to use. In still further contemplated aspects, the compounds according to the inventive subject matter may also be administered as recombinant nucleic acid in a manner that allows expression of the compound in a host cell. For example, recombinant nucleic acids may be provided to the target tissue via adenoviral vectors, transfection using lipids or liposomes, electroporation, or other manners well known in the art.

Examples of suitable aqueous and non-aqueous carriers which may be employed in the pharmaceutical compositions include water, ethanol, polyols (e.g., glycerol, propylene glycol, polyethylene glycol, etc.), and suitable mixtures thereof, vegetable oils, and injectable organic esters, such as ethyl oleate. Proper fluidity can be maintained, for example, by the use of coating materials, such as lecithin, by the maintenance of the required particle size in the case of dispersions, and by the use of surfactants. Most typically, suitable fluids are sterile and buffered to maintain a pH appropriate for stability of the active ingredient and site of injection or other use.

Therefore, it should be recognized that numerous compounds and compositions and particularly pharmaceutical compounds and compositions are contemplated that are effective to interact with at least one component of a signaling pathway associated with GDF-11 to thereby affect differentiation of retinal and auditory progenitor cells. The term “affect differentiation” as used herein refers to a change of the developmental the fate of a cell (i.e., its phenotypic and differentiated characteristics that define its function), and/or various aspects of cellular differentiation (e.g., changes in length of cell cycle, extension of axons and dendrites, elaboration of cellular processes or signaling machinery that enable it to communicate with other neural cells, and/or the environment). Thus, compositions are contemplated that comprise a compound at a concentration effective to interact with at least one component of a signaling pathway associated with GDF-11, GDF-8, and/or activins to thereby affect differentiation of a visual, auditory, and/or sensory neural progenitor cells. In especially preferred compositions, differentiation of the progenitor cell is affected with a concurrent change in cell proliferation of less than 5% (as compared to negative control).

Thus, especially contemplated are methods of enabling modulation of susceptibility of sensory/neural progenitor cells to a developmental stimulus by providing a composition that includes at least one of a GDF-11, a GDF-11 analog, and a GDF-11 antagonist in a pharmaceutically acceptable formulation. Preferably, information is provided in such methods to administer the composition to the sensory/neural progenitor cell (in vitro or in vivo) at a dosage and under a protocol effective to modulate the susceptibility of the progenitor cell, wherein the modulation of the susceptibility is maintained under the protocol for a period effective to increase or decrease a number of differentiated functional and/or neural cells derived from the progenitor cell.

Most typically, the dosage and protocol in such methods are established following experimental conditions for modulation as described below. Therefore, dosage will typically be adjusted such that the affected progenitor cells are contacted with contemplated compounds in a concentration range of about 0.01 ng/ml to about 1 mg/ml ( and in rare cases even higher), However, and more typically, suitable concentration ranges will be between about 0.1 ug/ml to about 100 ug/ml. Similarly, the protocol will typically follow the administrations as described below and it is generally contemplated that the compounds according to the inventive subject matter are administered over a period of at least 6 hours, more typically at least 24 hours, and most typically at least 2 days. Administration may be continuous (e.g., via drug-eluting implant) or in one or more dosage units (e.g. injection). As already discussed above, a person of ordinary skill in the art will be readily able to determine the appropriate dosage and schedule based various readily quantifiable parameters (e.g., determination of Math5 expression via quantitative PCR, or determination of changes in Smad2/3 phosphorylation in auditory or ocular cells via biopsy/animal model, etc.).

Therefore, and especially where contemplated compositions and methods are used in a pharmaceutical context, pharmaceutical kits for treatment of neural disorders that are characterized in their responsiveness to follistatin are especially contemplated. Such kits will typically include at least one of a GDF-11, a GDF-11 analog, and a GDF-11 antagonist in a pharmaceutically acceptable formulation and an instruction associated with the formulation wherein the instruction pertains to administration of the formulation to a sensory/neural progenitor cell at a dosage and under a protocol effective to modulate the susceptibility of the sensory/neural progenitor cell. Most preferably, the protocol is descriptive of a protocol that is effective to maintain modulation of the susceptibility for a period sufficient to increase or decrease a number of differentiated cells derived from the sensory/neural progenitor cell. Viewed from yet another perspective, use of at least one of a GDF-11, a GDF-11 analog, and a GDF-11 antagonist is contemplated in the manufacture of a medicament for treatment of an auditory or visual neural disorder, wherein the disorder is follistatin responsive.

### Experiments

Among other factors and considerations, the following experiments and observations lead the inventors to conclude that GDF-11 controls the period during which neural, and especially visual and auditory progenitor cells are competent to produce certain progeny, thus governing the relative numbers of sensory and/or neural cell types that arise. This discovery is particularly noteworthy as heretofore known activities of GDF-11 are in stark contrast to the findings presented herein. For example, in olfactory epithelium (OE), GDF-11 is known to negatively regulate neuron number by causing cell cycle arrest of the progenitor cells that give rise to olfactory receptor neurons (ORNs) (H. H. Wu et al., Neuron 37, 197 (2003)).

### Animals

CD-1 outbred mice (Charles River) and C57B16/J inbred mice (Jackson) were used to maintain various strains and for tissue culture experiments. For staging, midday of the day of vaginal plug discovery was designated embryonic day 0.5 (E0.5), and day of birth was considered postnatal day 0 (P0). For birthdating and cell proliferation analysis with bromodeoxyuridine (BrdU), pregnant dams were injected intraperitoneally with BrdU (50 μg/g body weight) and euthanized at indicated times thereafter. All protocols for animal use were approved by the Institutional Animal Care and Use Committee of the University of California, Irvine and were in accordance with NIH guidelines. GDF-11 null (Gdf11^{tm2/tm2}) mice were obtained by intercrossing Gdf11^{+/tm2} mice as described (H. H. Wu et al., Neuron 37, 197 (2003)). Fst^{-/-} mice were obtained by intercrossing Fst^{+/-} mice maintained on C57BL/6J background (M. M. Matzuk et al., Nature 374, 360 (1995)).

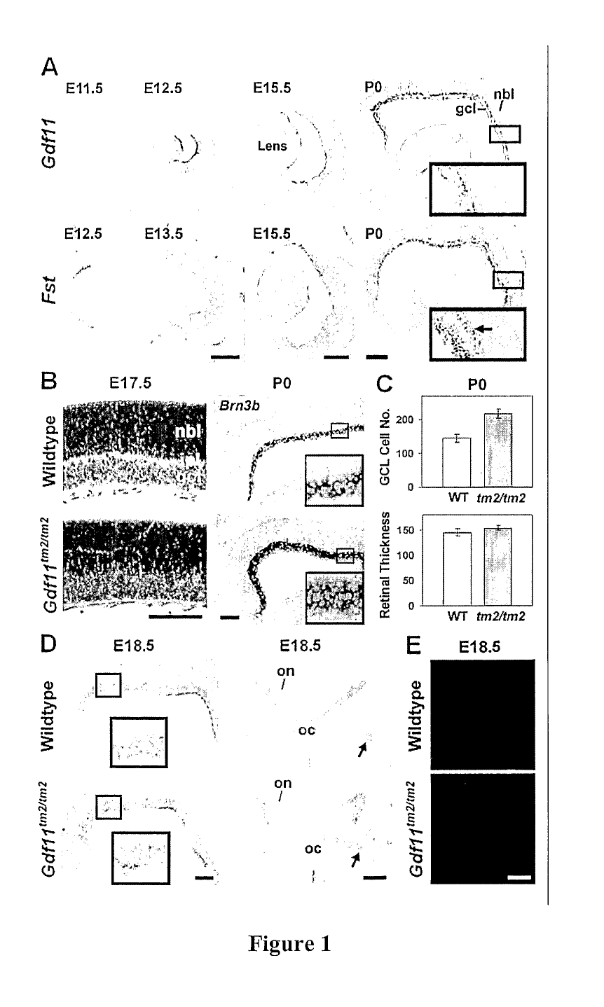

Gdf11^{+/-2}; Fst^{+/-} mice were generated by crossing Gdf11^{+/tm2} females with Fst^{+/-} male animals. Double null mutants (Gdf11^{tm2/tm2}; Fst^{-/-}) were obtained by intercrossing the resulting Gdf11^{+/tm2}; Fst^{+/-} mice. The Tattler 1 transgenic reporter mouse line, expressing a Tα1 tubulin promoter-driven tau-lacZ fusion gene, was generated as part of a series of reporter mice previously described (R. C. Murray, D. Navi, J. Fesenko, A. D. Lander, A. L. Calof, J Neurosci 23, 1769 (2003)). Tattler 1 mice express the tau-β-galactosidase fusion reporter protein in the cell bodies and axons of RGCs (FIG. 1D and A. D. Lander, unpublished observations). Gdf11^{+/tm2}; Tattler-1 animals were mated with Gdf11^{+/-2} animals to generate Gdf11^{tm2/tm2}; Tattler-1 and Gdf11^{+/+}; Tattler-1 littermate embryos for analysis.

### Tissue Culture

Neural retinas from E13.5 CD-1 mouse embryos were dissected free of surrounding ocular tissue and lens. Whole neural retinas were place into Millicell chambers (filter pore size 0.45 μm, Millipore) in 24 well plates and incubated for 2, 3 or 4 days in DMEM/F12 (1:1, Invitrogen/Gibco) containing insulin (20 μg/ml), human transferrin (100 μg/ml), progesterone (60 ng/ml), putrescine (16 μg/ml), selenium (40 ng/ml), and 5% heat-inactivated fetal bovine serum. Recombinant human GDF-11 (50 ng/ml, obtained by agreement with Wyeth Research) was added daily. Significance of Math5 reduction in GDF-11 treated explants was confirmed by a blind test with 6 individuals ranking ISH images of 5 untreated (control) and 5 GDF-11-treated retinas (p<0.05, Mann-Whitney Rank-Sum test).

### In situ hybridization (ISH), Immunofluorescence, Histological Analysis

Embryos, dissected eyes plus optic nerves, or retinal explants were fixed in 4% paraformaldehyde in phosphate buffered saline (PBS) and cryoprotected in 30% sucrose/PBS. Embedded animals and explants were sectioned on a cryostat at 20 μm for ISH and 12 μm for immunohistochemistry. ISH using digoxigenin-labeled cRNA probes was performed as described (H. H. Wu et al., Neuron 37, 197 (2003)). Probes used in this study were generated from the following: 1.2 kb mouse GDF-11 partial cDNA, 318 bp mouse Fst partial cDNA, 679 bp mouse Brn3b partial cDNA (bp 266-945 of Genbank #NM138944), 644 bp mouse Crx1 partial cDNA (bp 482-1126 of Genbank #NM007770), 389 bp mouse Math5 partial cDNA (bp 1-390 of Genbank #AF071223), 2.0 kb mouse Mash1 full-length cDNA, 349 bp mouse NeuroD partial cDNA, 445 bp mouse Alk4 partial cDNA (bp 31-476 of Genbank #NM007395), 424 bp mouse Alk5 partial cDNA (bp 87-511 of Genbank #NM009307), 401 bp mouse ActRIIa partial cDNA (bp 71-472 of Genbank #M65287), 308 bp mouse ActRIIb partial cDNA (bp 119-427 of Genbank #M84120), 741 bp mouse Chx10 partial cDNA (bp 934-1675 of Genbank #NM007701), 302 bp mouse Pax6 partial cDNA, 656 bp mouse Rax partial cDNA (bp 28-683 of Genbank #NM013833), 803 bp mouse Six6 partial cDNA (bp 832-1635 of Genbank #NM011384), 607 bp mouse Vax2 partial cDNA (bp 504-1111 of Genbank #NM011912), 842 bp mouse Math3 partial cDNA (bp 129-971 of Genbank #NM007501), 1412 bp mouse Ngn2 partial cDNA, 1037 bp rat Hes1 partial cDNA, 735 bp mouse Foxn4 partial cDNA (bp 651-1386 of Genbank #NM148935), 300 bp mouse Prox1 partial cDNA, 687 bp of mouse Otx2 partial cDNA (bp 894-1581 of Genbank #NM 144841). Similar results were obtained from multiple sections of multiple animals (n=2-4 litter pairs) or explants (n=6-10 explant pairs (±GDF-11 treatment) from two independent experiments) for each probe.

For immunofluorescence studies, cryosections were blocked in 10% bovine calf serum/0.1% Triton-X 100 in PBS for 1 hour, incubated overnight at 4° C. with mouse anti-Syntaxin (1:1000 dilution of ascites fluid, Sigma), mouse anti-Neurofilament 68 (1:500 dilution of ascites fluid, Sigma), or mouse anti-Lim1/2 (1:100, Developmental Studies Hybridoma Bank), and detected with Texas Red-conjugated goat antimouse IgG (1:100, Jackson). Cells in M-phase were detected by immunostaining using polyclonal rabbit anti-phospho-histone H3 (Upstate Biotechnology, Cat. No. 06-570) at 1:200 dilution, visualized with Alexa Red-conjugated goat anti-rabbit-IgG (1:1000 dilution; Molecular Probes). Cell nuclei were counterstained with Hoechst 33342 (10 μg/ml). For histological analysis, embryos were fixed in Bouin's fixative for 24 hours, processed for paraffin sectioning (10 μm), and stained with hematoxylin-eosin.

### BrdU Labeling in vivo

Cryosections (12 μm) were processed for anti-BrdU immunoreactivity as described (R. C. Murray, D. Navi, J. Fesenko, A. D. Lanader, A. L. Calof, J Neurosci 23, 1769 (2003)). For RGC birth-dating, pregnant dams were given two injections of BrdU (at 1 hour intervals) at E13.5 or E15.5, then euthanized 48 hours later. Double labeling of Brn3b and BrdU was performed by detecting Brn3b transcripts with ISH, which strips histones from DNA, followed by BrdU immunohistochemistry as described.

### β-Galactosidase Histochemistry

E18.5 embryos were fixed in 2 mM MgCl2, 4% paraformaldehyde in 0.02 M NaPO4, 0.15 M NaCl, pH 7.5 for 2 hr at room temperature, cryoprotected in 30% sucrose/PBS, and sectioned at 30 μm on a cryostat. Sections were stained in 1 mg/ml 5-bromo-4-chloro-3-indolyl-β-d-galactopyranoside (X-gal), 5 mM K3Fe(CN)6, 5 mMK4Fe(CN)6, 2 mM MgCl2, 0.1% Triton X-100, 0.01% deoxycholate, in PBS at 37° C. as described elsewhere.

### Western Blotting

Neural retinas from Gdf11^{tm2/tm2} embryos or wildtype littermates were dissected free of surrounding ocular tissue and lens at E17.5, and lysed directly in SDS gel loading buffer. Proteins were separated on 12% SDS-PAGE and transferred to PVDF membrane (Millipore) using standard procedures. The membrane was incubated with mouse antip27Kip1 (1:500, Neomarker clone DCS-72.F6) for 2 hr and incubated with horseradish peroxidase-conjugated anti-mouse IgG (1:5000, BioRad) for 1 hr. After washing, the membrane was dipped in chemiluminescence substrate and exposed to Hyperfilm (Amersham). The blot was then stripped in 100 mM Tris, pH 7.4, 2% SDS, 100 mM β-mercaptoethanol for 30 min at 70 ° C., rinsed, and re-probed with rabbit antiactin (1:200, Sigma) as a control.

### Experimental Results and Observations

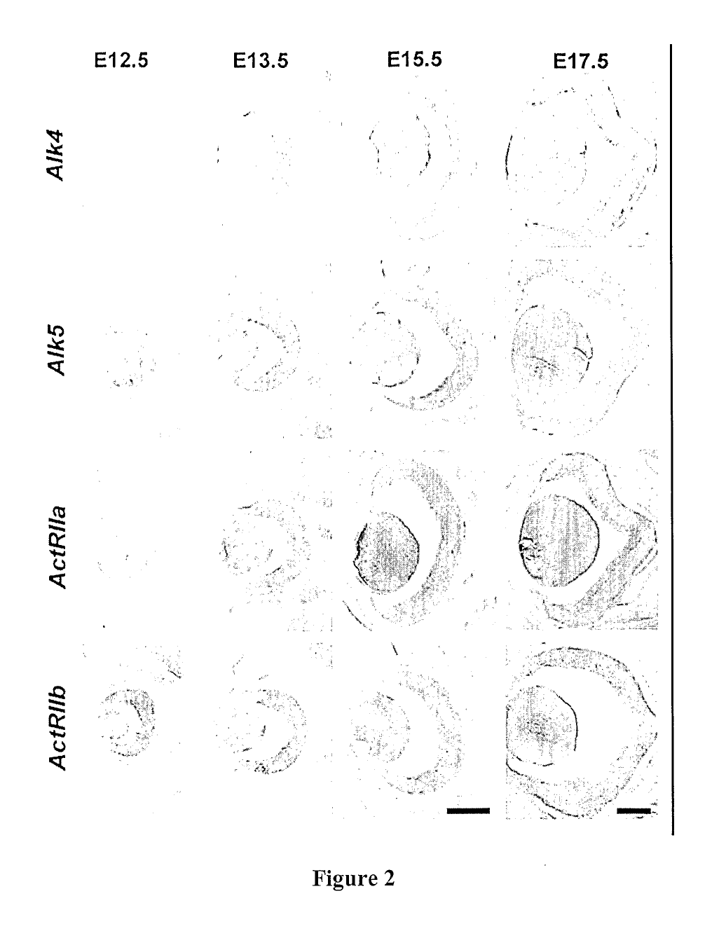

In mouse retina GDF-11 expression begins about E12.5, when RGCs begin to differentiate as can be seen from FIG. 1A. GDF-11 mRNA is observed throughout the retina, including the neuroblastic layer (NBL), until at least the first postnatal day (P0), although by E15.5, expression is highest in the developing ganglion cell layer (GCL). Expression of follistatin (Fst), which encodes a secreted GDF-11 antagonist (L. W. Gamer et al., Dev Biol 208, 222 (1999)), is first detected at E13.5. From E15.5 on, Fst expression is highest in the nascent GCL, but also evident in the NBL and presumptive amacrine cells. Putative receptors for GDF-11 (H. H. Wu et al., Neuron 37, 197 (2003), S. P. Oh et al., Genes Dev 16, 2749 (2002), S. J. Lee, A. C. McPherron, Proc Natl Acad Sci USA 98, 9306 (2001), and S. M. Federman et al., Journal of Bone Mineral Research 15, S103 (2000)) are also expressed in appropriate patterns in the neural retina from E12.5-13.5 onward. FIG. 2 depicts ISH analysis of presumptive GDF-11 receptor expressions during retinal development. Expression of type I receptor Alk4 (ActR1B) and activin type II receptor ActRIIa is detected throughout neural retina after E13.5, being somewhat more prominent in the ganglion cell layer adjacent to the chamber. Transcripts of Alk5 (TGB-β Type I receptor) and ActRIIb (activin type II receptor B) appear to be distributed evenly in the neural retina at all stages examined. No clear changes in the levels of the receptor expression are apparent after E13.5. Thus, the availability of receptors does not seem to be a limiting factor for GDF-11 action. Scale bar, 200 μm.

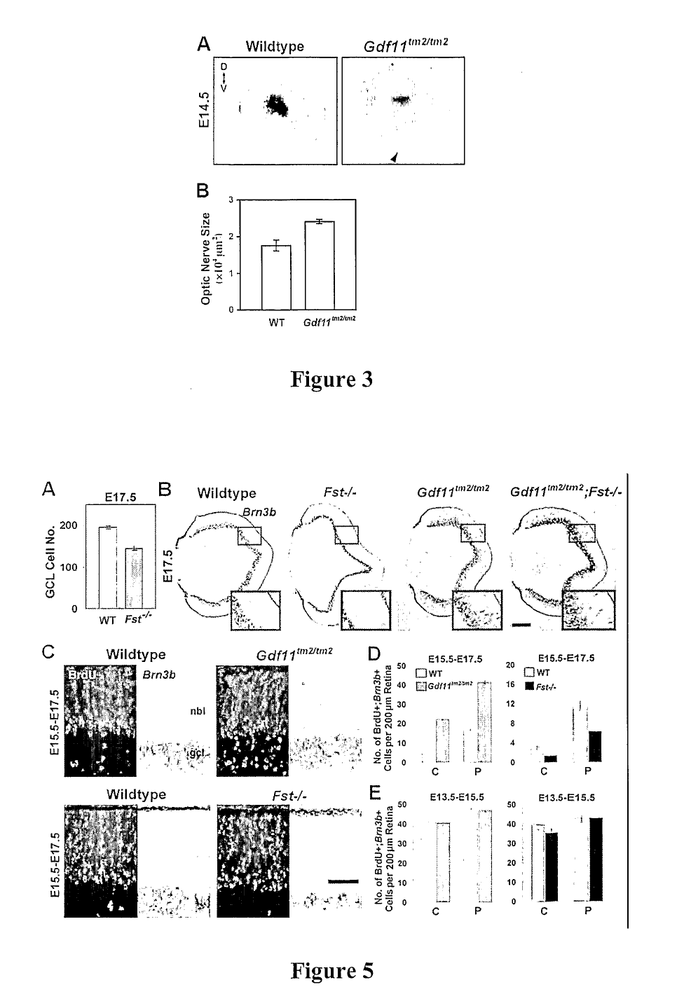

To investigate the role of GDF-11 in retinal development, the inventors examined mice homozygous for the null allele Gdf11^{tm2} (H. H. Wu et al., Neuron 37, 197 (2003)). Gdf11^{tm2/tm2} retinas show obvious changes as early as E14.5, when closure of the optic fissure is incomplete as can be seen in FIG. 3: (A) Retinal abnormalities in Gdf11^{tm2/tm2} mice included incomplete closure of the optic fissure (arrowhead) at E14.5; in wildtype animals, closure occurs around E12.5. D, dorsal; V, ventral. After E16.5, the optic fissure closes in Gdf11^{tm2/tm2} retinas, and subsequent formation of optic disc, optic nerve and periocular tissue appear to be unaffected. (B) Quantification of neurofilament-stained optic nerve sections shown in FIG. 1E. Cross sections (20 μm) of dissected optic nerves 0.8-1.2 mm from the optic disc were stained with anti-neurofilament antibodies as described in Methods, and section area measured using NIH Image (7 sections per animal, 3 animals of each genotype). Mean areas: Gdf11^{tm2/tm2}, 24,082.26 (±641.84 SEM) μm2; wildtype, 17,562.14 (±1525.9 SEM) μm2. By E17.5, the presumptive GCL of mutant embryos has an abnormally high cell density, and the inner plexiform layer (IPL), well demarcated in wildtype littermates, is not observed as shown in FIG. 1B.

Increased cell density in the mutant GCL is accompanied by widening of the cell layer expressing Brn3b (Gdf11^{tm2/tm2}, 49.5±3.3 μm; wildtype, 38.5±0.4 μm [SD]), which encodes a POU-domain transcription factor specific for differentiated RGCs. By P0, the latest time at which the mutant is viable, Gdf11^{tm2/tm2} GCLs contain ˜50 % more cells than wildtypes as can be taken from FIG. 1C. The excess RGCs that form in Gdf11^{tm2/tm2} animals appear to differentiate normally, extending axons through the optic chiasm and tracts, which also appear abnormally thick as illustrated in FIG. 1D. By neurofilament immunohistochemistry, the inventors estimate a 37% increase in the cross-sectional areas of optic nerves in Gdf11^{tm2/tm2} animals as shown in FIG. 1E and FIG. 3.

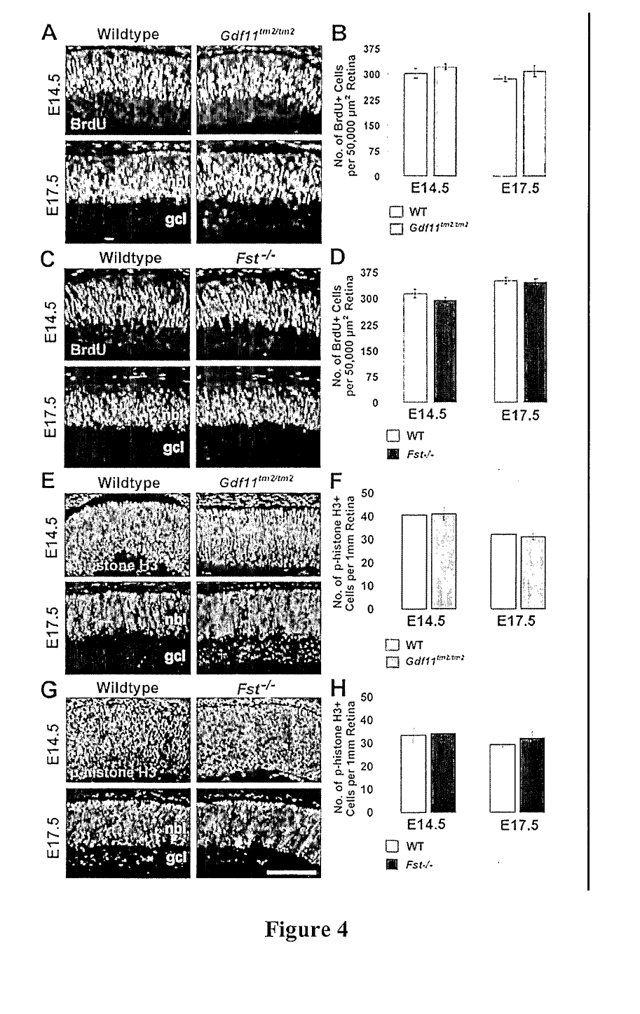

These changes, observed in all mutant mice examined (>32), imply that GDF-11 is a negative regulator of RGC genesis. In this respect, the changes in Gdf11^{tm2/tm2} retinas recall those in OE, in which Gdf11^{tm2/tm2} mice also have excess differentiated ORNs. However, unlike the situation in OE, Gdf11^{tm2/tm2} retinas display no increase in overall thickness, nor are the distribution nor number of proliferating cells significantly altered as can be seen in FIG. 4: Normal cell proliferation pattern in Gdf11^{tm2/tm2} and Fst^{-/-} retinas. (A-D) Proliferating cells were labeled by a single in vivo injection of BrdU into pregnant dams at 1 hr (for Fst^{-/-}) or 2 hr (for Gdf11^{tm2/tm2}) prior to sacrifice at E14.5 or E17.5. Cells that incorporated BrdU were visualized by immunostaining (red). Cell nuclei were counterstained with Hoechst (blue). (B and D) Quantitative analyses of BrdU labeling. There are no significant changes in the distribution or numbers of BrdU-labeled cells in either Gdf11^{tm2/tm2} or Fst^{-/-} retinas. (E-H) Phosphorylated-histone H3 (p-histone H3) immunostaining. Anti-p-histone H3 antibody recognizes cells in late G2 and M phase (red). Mitotic nuclei are only detected at the outer margin of the neural retina, the retinal ventricular zone (11). (F and H) Quantitative analysis of p-histone H3 staining. Both pattern and number of p-Histone H3-stained cells are the same in Gdf11^{tm2/tm2} and Fst^{-/-} retinas as in wildtypes. Histograms show mean±SEM; n=3 animals of each genotype nbl, neuroblastic layer; gcl, ganglion cell layer. Scale bar, 100 μm.

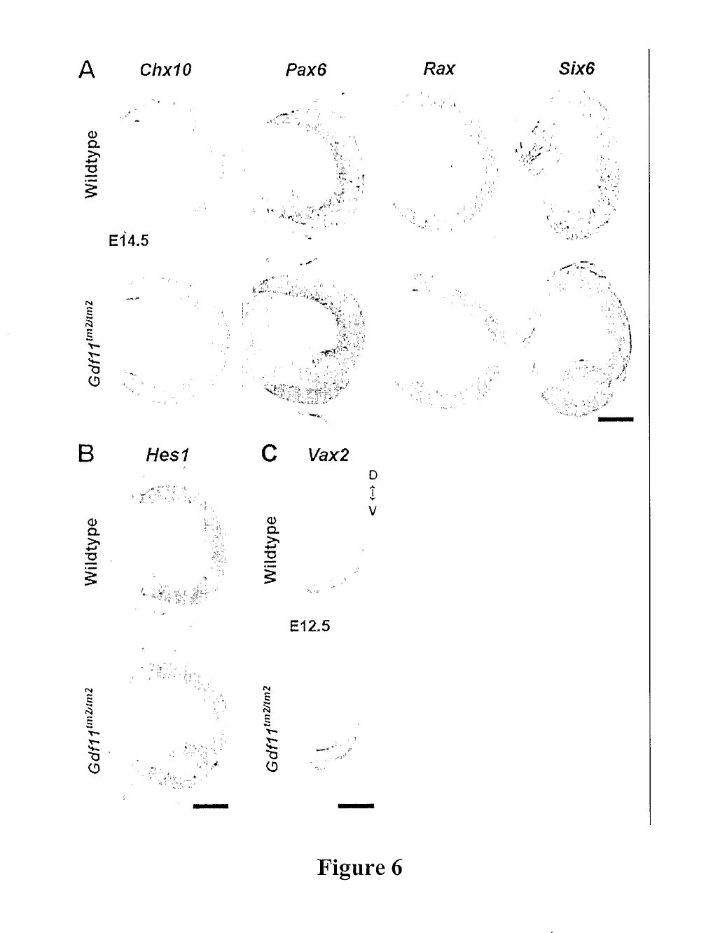

These observations clearly indicated that the mechanism by which GDF-11 regulates neurogenesis in the retina significantly differs from that in the OE. Since Fst is known to antagonize GDF-11 function in vivo and in vitro (L. W. Gamer et al., Dev Biol 208, 222 (1999)), the inventors also examined Fst^{-/-} and Gdf11^{tm2/tm2}; Fst^{-/-} retinas. Fst^{-/-} retinas showed a 26% reduction in the number of cells in the GCL and a large decrease in thickness of the Brn3b⁺ cell layer as shown in FIGS. 5A and 5B, indicating that Fst is a positive regulator of RGC development. Gdf11^{tm2/tm2}; Fst^{-/-} retinas showed an expanded Brn3b⁺ GCL, comparable to that observed in Gdf^{tm2/tm2} retinas, consistent with the primary role of Fst being to inhibit GDF-11 (see FIG. 5B). Just as in Gdf11^{tm2/tm2} retinas, the level and pattern of progenitor cell proliferation was unaltered in Fst^{-/-} retinas as can be seen in FIG. 4. The fact that cell proliferation is normal in Gdf11^{tm2/tm2} and Fst^{-/-} retinas suggests that the size of the progenitor pool is not regulated by GDF-11. Moreover, expression of several genes involved in early eye specification, patterning, and expansion is also normal in Gdf11^{tm2/tm2} mice (see FIG. 6: Normal expression of key regulatory and ventral patterning genes in Gdf11^{tm2/tm2} retinas. (A) Expression of Chx10, Pax6, Rax, and Six6 appears to be unaltered in Gdf11^{tm2/tm2} retinas at E14.5. These genes are known to be required for both eye specification and progenitor cell proliferation early in development. Normal expression of these genes suggests that initial expansion of retinal progenitor cells is properly regulated in Gdf11^{tm2/tm2} retinas. (B) Expression of the bHLH repressor gene, Hes1 expression, appears normal in Gdf11^{tm2/tm2} retina at E14.5, suggesting that progenitor cells do not undergo premature differentiation. (C) Vax2, a gene involved in ventral patterning of developing retina, is expressed normally at E12.5, suggesting that the delayed optic fissure closure in Gdf11^{tm2/tm2} animals is not caused by a failure in retinal ventralization. D, dorsal; V, ventral. Scale bars, 200 μm.).

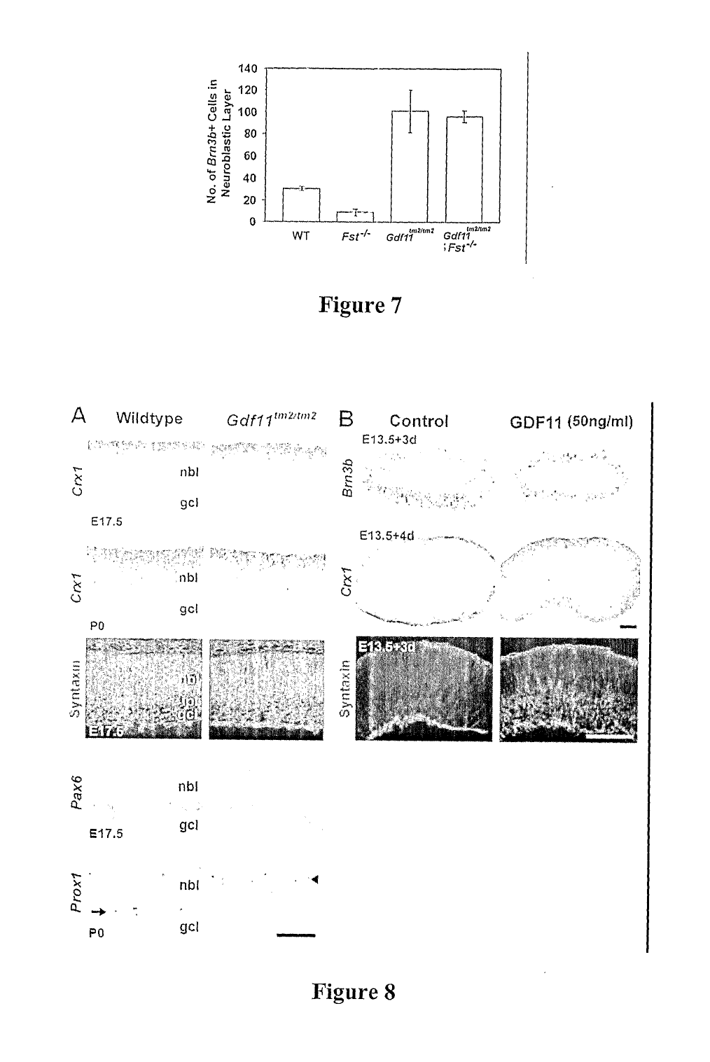

During development, RGCs are born at the outer margin of the neural retina and migrate inward to the GCL during a defined period. Detailed examination of Gdf11^{tm2/tm2} and Gdf11^{tm2/tm2}; Fst^{-/-} retinas at E17.5 revealed that the NBL of these mutants contains three times as many Brn3b⁺ cells (migrating RGCs) as wildtypes (see FIG. 5B insets, and FIG. 7, depicting a quantitative analysis of Brn3b ISH shown in FIG. 5B. The number of Brb3b⁺ cells in the NBL is decreased in Fst^{-/-} retinas. In contrast, the number is significantly increased in both Gdf11^{tm2/tm2} and Gdf11^{tm2/tm2}; Fst^{-/-} retinas. Note that double mutant retinas show an increase similar to that observed in Gdf11^{tm2/tm2} retinas. Histogram shows mean±SEM; n=2 animals of each genotype.). This suggested that, in Gdf11^{tm2/tm2} retinas, RGC production may be prolonged beyond its normal period. To test this, the inventors performed birthdating experiments. The results, shown in FIG. 5C, show an abnormally large number of BrdU+ cells in the GCL of Gdf11^{tm2/tm2} animals pulsed with BrdU from E15.5-E17.5. Conversely, BrdU+ cells in the GCL of Fst^{-/-} animals pulsed over this same timecourse were strongly decreased in number, as expected if Fst acts to inhibit GDF-11. These differences were not seen when pulse labeling was done at earlier ages (see FIG. 5E). Thus, although onset of RGC production appears unaffected by loss of GDF-11 or Fst, its downregulation is delayed in Gdf11^{tm2/tm2} retinas (and accelerated in Fst^{-/-}). A lengthened period of RGC production likely explains why Gdf11^{tm2/tm2} retinas accumulate abnormally large numbers of RGCs.

To determine whether GDF-11 regulates production of other retinal cell types, the inventors examined rod photoreceptors and amacrine cells, two cell types whose peak periods of differentiation follow that of RGCs. Crx1, a marker for early photoreceptors, is normally upregulated around birth when rod photoreceptor production peaks, and expands to cover much of the NBL. In Gdf11^{tm2/tm2} retinas, upregulation and expansion of Crx1 expression are not observed as can be seen in FIG. 8A. Amacrine cells may be visualized by expression of syntaxin, as well as Pax6 and Prox1. In the amacrine cell layer of Gdf11^{tm2/tm2} retinas, expression of all three markers was reduced as shown in FIG. 8A. Altogether, these results suggest that prolonged production of RGCs in Gdf11^{tm2/tm2} retinas occurs at the expense of cell types (amacrine cells, photoreceptors) that normally differentiate after RGC production has declined. No apparent increase in amacrine cell or photoreceptor production in Fst^{-/-} animals was observed, possibly because excess GDF-11 activity in Fst^{-/-} retina is mitigated by the reduction in RGC cells, which express the highest levels of GDF-11 (see FIG. 1).

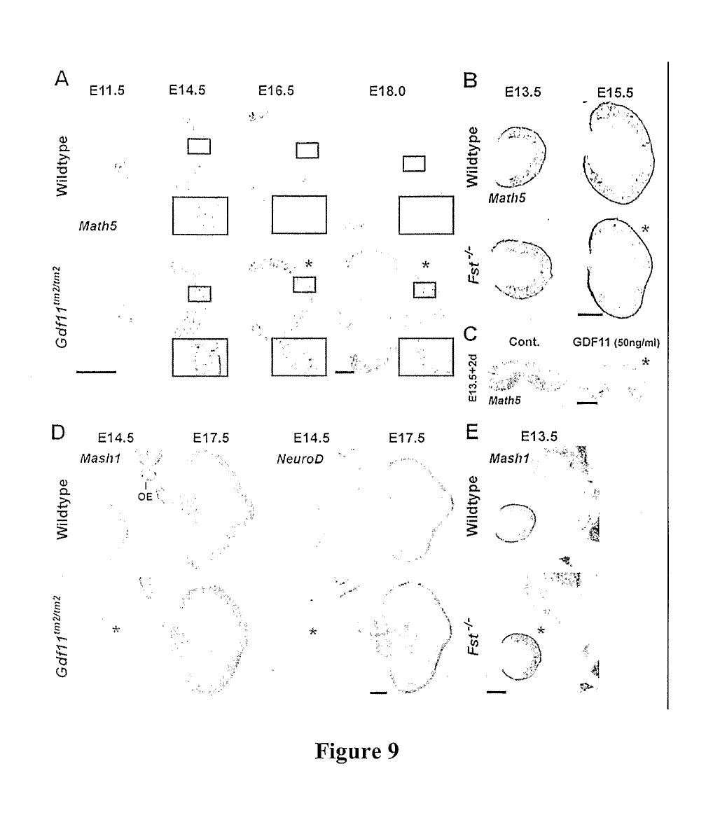

The inventors further tested the hypothesis that GDF-11 controls amacrine and photoreceptor cell number, as well as RGC number, by using retinal explant cultures to examine effects of exogenous GDF-11 on wildtype retinas. E13.5 retinal explants grown in GDF-11 exhibited a large reduction in Brn3b⁺ RGCs, whereas expression of both Crx1 (photoreceptor marker) and syntaxin (amacrine cell marker) were increased with GDF-11 treatment as seen in FIG. 8B. These findings support the hypothesis that GDF-11 is an important regulator of all three retinal cell types. The finding that RGC genesis is increased, while amacrine and rod production are decreased, in GDF-11 nulls led the inventors to hypothesize that GDF-11 regulates induction of cell-intrinsic changes by which progenitor cells lose competence to produce RGCs and acquire competence to produce later-born cell types. If GDF-11 directly controls progenitor cell competence, GDF-11 mutants might exhibit changes in expression of factors that determine competence states. Math5 is among the first such factors expressed during retinal neurogenesis, and is required for competence to produce RGCs (S. Kanekar et al., Neuron 19, 981 (1997); N. L. Brown, S. Patel, J. Brzezinski, T. Glaser, Development 128, 2497 (2001); Z. Yang, K. Ding, L. Pan, M. Deng, L. Gan, Dev Biol 264, 240 (2003)). Math5 expression is initiated normally in Gdf11^{tm2/tm2} retinas, but mutants maintain high levels of expression in the NBL for an abnormally long period: Normally, Math5 expression is downregulated in central NBL by E16.5, and is essentially absent by E18.0; in Gdf11^{tm2/tm2} retinas, however, Math5 expression is still evident at these ages as evident from FIG. 9A.

Conversely, downregulation of Math5 expression occurs prematurely in Fst^{-/-} retinas as can be seen in FIG. 9B, and is accelerated when retinal explants are cultured in GDF-11 as can be seen in FIG. 9C. The prolonged period of Math5 expression in Gdf11^{tm2/tm2} retinas corresponds to the period of prolonged RGC genesis (see FIG. 5). The alteration in the period of Math5 expression in Gdf11^{tm2/tm2} retinas is accompanied by a shift in onset of expression of two other proneural genes, Mash1 and NeuroD, which are involved in the development of bipolar and amacrine cells. In Gcf11^{tm2/tm2} embryos, expression of both genes is barely detectable at E14.5, when significant levels are seen in wildtypes as is apparent in FIG. 9D. Conversely, Mash1 expression occurs prematurely in Fst^{-/-} retinas, at E13.5, when wildtype littermates express only low levels of Mash1 (see FIG. 9E). By E17.5 both Mash1 and NeuroD expression recover to normal levels in Gdf11^{tm2/tm2} retinas (see FIG. 9D), suggesting that progenitor cells can acquire competence to produce later-born cell types even though Math5 expression (and RGC genesis) remain elevated. Altogether, these observations suggest that GDF-11 regulates the timing of progenitor competence by controlling the expression of genes crucial for progenitor cell fate determination.

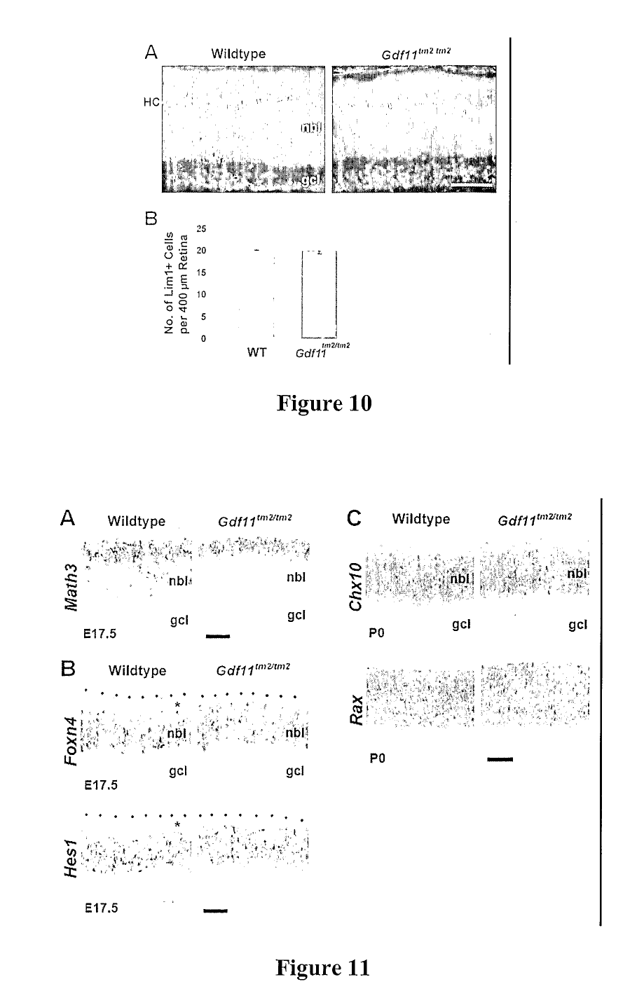

The inventors then examined whether GDF-11 regulates generation of all retinal cell types, or only selected cell types. Since Gdf11^{tm2/tm2} animals die at birth, this question cannot yet be answered with certainty. Expression of Lin1, a horizontal cell-specific transcription factor, appears to be normal in Gdf11^{tm2/tm2} retinas as illustrated in FIG. 10 (Expression of the horizontal cell-specific transcription factor, Lim1. (A) Horizontal cells were detected by immunostaining of cryosections with anti-Lim1/2 antibody at P0. (B) There was no significant alteration in the number of cells expressing Lim1 in Gdf11^{tm2/tm2} retinas at P0. Histogram shows mean±SEM; n=3 animals of each genotype. HC, horizontal cells. Scale bar, 50 μm.), although changes in expression of a number of other regulatory genes expressed by retinal progenitors are observed as shown in FIG. 11 depicting expression patterns of transcription factors implicated in retinal neurogenesis. (A) Abnormal expression of Math3, a gene involved in the development of amacrine and bipolar cells. In Gdf11^{tm2/tm2} retina, Math3 expression is restricted to the outer margin (ventricular layer) of the neural retina, suggesting that in the mutant, fewer progenitor cells have competence to develop into amacrine and bipolar cells. Scale bar, 50 μm. (B) Changes in the distribution of cells expressing Foxn4 and Hes1. Foxn4 and Hes1 are expressed in retinal progenitor cells and are downregulated in postmitotic neurons. Wildtype retinas show a clear Foxn4/Hes1 negative cell domain at the outer margin, where photoreceptors differentiate (red asterisks). This domain is reduced in Gdf11^{tm2/tm2} retinas, consistent with the idea that photoreceptor development may be decreased in mutant animals. Dotted line marks the border of neural retina. Scale bar, 50 μm. (C) Expression of genes involved in the development of bipolar cells and Müller glial cells is unchanged in Gdf11^{tm2/tm2} retinas. Expression of Chx10, a gene regulating bipolar cell development, and Rax, required for Müller glial cell development, are unaltered in mutants. Scale bar, 50 μm. In addition, expression of Ngn2 and Otx2 appear to be normal in the mutant retinas at all stages examined (data not shown). However, the absence of an effect on horizontal cells indicates that GDF-11 signaling does not regulate production of all cell types in the retina. Instead, it must govern either a specific subprogram of retinal neurogenesis, or act on only a subset of multipotent progenitor cells. This last idea suggests that early retinal progenitors, despite possessing the potential to give rise to all retinal cell types, are nonetheless heterogeneous, at least with respect to their capacity to respond to GDF-11.

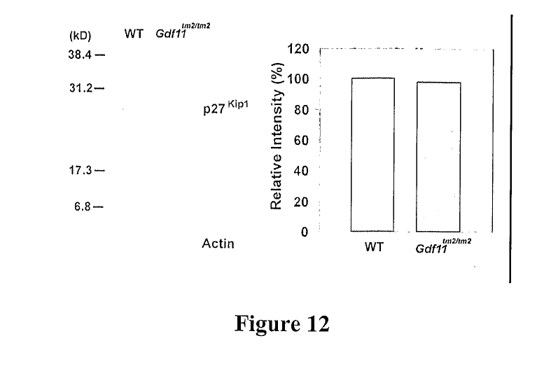

The inventors' finding that GDF-11 governs retinal progenitor cell fate without altering proliferation supports the theory that regulation of cell division and cell type determination occur independently in the retina (D. L. Turner, E. Y. Snyder, C. L. Cepko, Neuron 4, 833 (1990)). Moreover, the present results highlight the difference in feedback mechanisms employed in different regions of the developing nervous system to effect proper neuron number: In retina, feedback regulation of neural cell number, mediated by GDF-11 expressed by the earliest-bon neurons, is accomplished by altering the fates of multipotent progenitor cells independent of proliferation. In other regions, such as OE, neuronal GDF-11 feeds back to regulate progenitor cell proliferation, independent of changes in cell fate. Finally, the present results highlight the unexpected diversity of action of GDF-11 itself: In OE, GDF-11 exerts its antineurogenic action by inducing reversible cell-cycle arrest in committed progenitors via increased expression of the cyclin-dependent kinase inhibitor, p27Kip1. In retina, by contrast, GDF-11 controls the timecourse of expression of genes that regulate competence to produce RGCs, but neither p27Kip1 levels nor cell proliferation are affected as can be seen in FIGS. 4 and 12 wherein FIG. 12 depicts normal levels of p27Kip1 in Gdf11^{tm2/tm2} retina at E117.5. The level of p27Kip1 was examined by Western blotting. Intensity of the p27Kip 1 band was normalized to that of actin band. Densitometry showed no change in the relative intensities of the p27Kip1 bands. Thus, GDF-11 acts as a negative feedback regulator of neurogenesis during development, by altering either progenitor cell proliferation, or progenitor cell fate, in different tissues.

It should be particularly noted that both GDF-11 and follistatin are also expressed in various tissues other than visual neural tissue, and that the above-discussed considerations will likely also apply to at least some of these tissues. For example, the sensory vestibulo-cochlear epithelium (lining the semicircular canals and cochlea) that contains the hair cells and hair cell progenitors (i.e., cells that give rise to hair cells, by cell division and/or differentiation) has been shown to express GDF-11 and follistatin. Furthermore, GDF-11 and follistatin are also expressed in the spiral acoustic ganglion, whose processes form the vestibulo-cochlear nerve that connects the sensory cells of the vestibulo-cochlear epithelium with the brain, and follistatin is also expressed in the connective tissue, contained within the bony labyrinth of the temporal bone, that surrounds the semi-circular canals, cochlea, spiral-acoustic ganglion, and vestibulo-cochlear (auditory) nerve.

Remarkably, reduced numbers of cells in the vestibulo-cochlear sensory epithelium are observed in follistatin Fst^{-/-} mice (sapra), which are characterized by complete absence of follistatin. Moreover, both the spiral acoustic ganglion and the vestibulo-cochlear nerve are smaller in size in Fst^{-/-} mice. Based on analogy with the retinal system as described above, and to some degree also with the olfactory epithelium, and further known biochemical functions of GDF-11, it is contemplated that in the vestibulo-cochlear epithelium, GDF-11 controls expression of Math1. It should be noted that Math1 is a transcription factor whose expression has been documented elsewhere to confer competence to form hair cells of the vestibulo-cochlear epithelium. The inventors therefore contemplate that GDF-11 will control Math1 expression in the vestibulo-cochlear epithelium not by controlling the number of Math1-expressing progenitors (i.e., by controlling their division), but rather by controlling expression of p27Kip1, which defines a broader field of dividing sensory epithelial cells, out of which hair cells are selected during development by gaining expression of Math1. GDF-11 is known to control p27 expression by progenitor cells of the olfactory epithelium (Wu et al, 2003). Furthermore, the inventors' data in the inner ear of the Fst^{-/-} (not shown) indicate that the p27 domain of the cochlear epithelium is decreased in size in the absence of Follistatin (which would be expected as the antagonist of an antineurogenic factor [i.e. GDF11 and/or GDF-11 analogs] is removed).

Similarly, the inventors contemplate that in the spiral-acoustic ganglion, GDF-11 will control expression of Neurogenin 1, which is required for the formation of spiral acoustic ganglion neurons. Such effect may be mediated either be through direct control of expression of Neurogenin 1, or by controlling (inhibiting) division of Neurogenin 1-expressing neuronal progenitors, a function analogous to that observed for Neurogenin 1 expressing progenitors in the developing olfactory epithelium. Further data (not shown) indicate that the spiral acoustic ganglion in the Fst^{-/-} mouse is smaller, and has reduced expression of Ncam, a differentiated neuron marker, and also that the synaptic domain of spiral-acoustic ganglion neurons, onto the hair cells of the inner ear, is reduced in size.

Therefore, it is contemplated that GDF-11 and follistatin control both the number and differentiation of hair cells and hair cell progenitors in the inner ear, and the size and synaptic connectivity of the spiral acoustic ganglion and vestibulo-cochlear (auditory) nerve in a manner similar to the way they control sensory/neuronal cell number in the retina. As can be taken from the above considerations and experiments, various TGF-beta superfamily proteins and their agonists and antagonists, and particularly GDF-11 control the numbers of RGCs, as well as amacrine and photoreceptor cells that form during development. Remarkably, GDF11 does not affect proliferation of progenitors, but instead controls duration of expression of Math5, a gene that confers competence for RGC genesis, in progenitor cells. Thus, GDF11 and other TGF-beta superfamily proteins and their agonists and antagonists can be used to influence temporal windows during which multipotent progenitors retain competence to produce distinct neural progeny.

Thus, specific embodiments and applications of compositions/methods for treatment of neural disorders using transforming growth factor-beta superfamily proteins and their antagonists have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. A method of enabling modulation of susceptibility of a neural progenitor cell to a developmental stimulus, comprising:
providing a composition that includes at least one of a GDF-11, a GDF-11 analog, and a GDF-11 antagonist in a pharmaceutically acceptable formulation;
instructing a person to administer the composition lo the neural progenitor cell at a dosage and under a protocol effective to modulate the susceptibility of the neural progenitor cell; and
wherein the modulation of the susceptibility is maintained under the protocol for a period effective to increase or decrease a number of differentiated neural cells derived from the neural progenitor cell.

2. The method of claim I wherein the neural progenitor cell is a progenitor cell for cells associated with visual or auditory function.

3. The method of claim 2 wherein the neural progenitor cell is a cell giving rise to at least one of a retinal ganglion cell, an amacrine cell, a rod photoreceptor cell, a cone photoreceptor cell, a ciliary body cell, retinal pigmented epithelium cell, an inner hair cell, a outer hair cell, a supporting cell of a vestibulo-cochlear epithelium, a spiral acoustic ganglion neuron, and a vestibulo-cochlear nerve cell.

4. The method of claim 1 wherein modulation of the susceptibility is mediated by expression of a gene selected from the group consisting of Math5, Math1, and Neurogenin-1.

5. The method of claim 1 wherein administration of at least one of the GDF-11 and the GDF-11 analog results in a decrease of retinal ganglion cells derived from the progenitor cell.

6. The method of claim 1 wherein administration of the GDF-11 antagonist results in an increase of retinal ganglion cells derived from the progenitor cell.

7. The method of claim 1 wherein administration of at least one of the GDF-11 and the GDF-11 analog results in an increase of at least one of a photoreceptor cell and an amacrine cell derived from the progenitor cell.

8. The method of claim 1 wherein administration of the GDF-11 antagonist results in a decrease of at least one of a photoreceptor cell and an amacrine cell derived from the progenitor cell.

9. The method of claim 1 wherein the GDF-11 antagonist is follistatin, and wherein the GDF-11 analog is GDF-8 or an activin.

10. The method of claim 1 wherein at least one of the GDF-11, the GDF-11 analog, and the GDF-11 antagonist is recombinant and produced in situ in neural tissue.

11. The method of claim 10 wherein the at least one of the GDF-11, the GDF-11 analogs and the GDF-11 antagonist are produced from a viral genome.

12. A pharmaceutical kit for treatment of a neural disorder that is characterized in responsiveness to follistatin, comprising:
at least one of a GDF-11, a GDF-11 analog, and a GDF-11 antagonist in a pharmaceutically acceptable formulation;
an instruction associated with the formulation wherein the instruction pertains to administration of the formulation to a neural progenitor cell at a dosage and under a protocol effective to modulate the susceptibility of the neural progenitor cell; and
wherein the protocol is descriptive of a protocol that is effective to maintain modulation of the susceptibility for a period sufficient to increase or decrease a number of differentiated cells derived from the neural progenitor cell.

13. The pharmaceutical kit of claim 12 wherein The GDF-11 analog is GDF-8 or an activin, and wherein the GDF-11 antagonist is follistatin.

14. The pharmaceutical kit of claim 12 wherein modulation of susceptibility is described as modulation of expression of a gene selected from the group consisting of Math5, Neurogenin-1, and Math1.

15. The pharmaceutical kit of claim 12 wherein the progenitor cell is a progenitor cell for cells associated with visual or auditory function,

16. The pharmaceutical kit of claim 12 wherein the differentiated cells are selected from the group consisting of retinal ganglion cells, amacrine cells, photoreceptor cells, and hair cells.

17. Use of at least one of a GDF-11, a GDF-11 analog, and a GDF-11 antagonist in the manufacture of a medicament for treatment of an auditory or visual neural disorder, wherein the disorder is follistatin responsive

18. The use of claim 17 wherein at least one of the GDF-11, the GDF-11 analog, and the GDF-11 antagonist is a recombinant protein.

19. The use of claim 17 wherein the disorder is selected from the group consisting of macular degeneration, retinal ganglion degeneration, Leber's congenital amaurosis, and sensorineural hearing loss,

20. The use of claim 17 wherein the follistatin responsive disorder is characterized by exacerbation of the state of disorder upon administration of a compound that elevates or reduces an amount of follistatin present in a patient.
